(19) United States
(12) Patent Application Publication
(10) Pub. No.: US 2006/0229543 A1
(43) Pub. Date: Oct. 12, 2006

(54) Wrench for reducing femur midshaft fractures

(76) Inventor: Ignacio J. Calvo, Miami, FL (US)

Correspondence Address: RUBEN ALCOBA, ESQ., 3399 NE 72 AVENUE, SUITE211, MIAMI, FL, 33122, US

(21) Appl. No.: 11/079,856

(22) Filed: Mar. 13, 2005

(51) Int. Cl. A61F 5/00

(52) U.S. Cl. 602/39

(57) ABSTRACT

A device that aids in the alignment of displaced femoral fragments prior to the insertion of an intramedullary rod in a patient.

### BACKGROUND

In orthopedic practice, the current method of reducing a displaced fracture of the midshaft of the femur consists of manipulating the fragments of the femur by hand and traction to align the fragments, thereby permitting the insertion of an intramedullary rod.

Inherent problems in performing the above method, when dealing with patients that are of a heavy physical build, is that surgeons tend to struggle aligning the fragments of the femur. The problem sometimes requires the surgeon to open the fracture site in order to directly manipulate the bone fragments into alignment. This is undesirable, for many other complications can arise as a consequence of opening the fractured site.

The inventor realized that he needed to invent a tool that would allow him to easily manipulate and align the fractured ends of the femur so that an intramedullary rod can be inserted without having to open the fracture site.

The inventor using a derivation of a tool that was used by Hugh Owen Thomas to correct deformities in limbs of patients has invented a device that will allow him to align displaced femoral fragments.

### SUMMARY

The present invention is directed to a device that aids in the alignment of displaced femoral fragments prior to the insertion of an intramedullary rod in a patient. The medical device for reducing a fracture of the midshaft of a femur comprises a shaft, having a first and second ends, the shaft has a plurality of openings running along its length. A fixed arm connects perpendicularly to the first end of the shaft. A movable arm mounts on the shaft, wherein the movable arm is parallel and immediately above the fixed arm. A lever, having two ends and a midpoint, attaches to the shaft perpendicularly to the fixed arm, the shaft attaches to the lever's midpoint. Lastly, a locking means for locking the movable arm in a locked position. The locking means may be a pin that slides into at least one of the openings of the shaft.

A surgeon uses the device by first placing the device on a patient so that the fixed arm of the device is placed at the thigh in a position that is posterior to the level of a fracture site and the movable arm is placed in a position that is anterior to the level of the fracture site. Then the surgeon moves the moveable arm of the device to a position that minimizes the distance between the fixed and the movable arm while embracing the thigh of the patient. The surgeon then locks the movable arm in the position that minimizes the distance between the fixed and the movable arm. Then the surgeon moves the lever of the device so that the fragments at the fracture site are aligned. And lastly, the surgeon performs the known steps required to insert the intramedullary rod into the medullary canal of the femur of the thigh.

An object of this invention is to permit a surgeon to align displaced femoral fragments, thereby permitting the insertion of an intramedullary rod without having to open the fracture site.

A further object of this invention is to allow surgeons to align displaced femoral fragments without having to struggle when manipulating the femoral fragment. The present invention is a tool that uses a torque principle to reduce the amount of energy a surgeon has to exert when aligning the fractured ends of the femur.

### DRAWINGS

These and other features, aspects, and advantages of the present invention will become better understood with regard to the following description, appended claims, and accompanying drawings where:

FIG. 1 shows a perspective view of an embodiment of the present invention;

FIG. 2 shows a frontal view of the present invention;

FIG. 3 shows a side view of the present invention;

FIG. 4 shows the present invention on a patient while it is being manipulated; and

FIG. 5A-5C show how the present invention aligns the fracture site of a femur.

### DESCRIPTION

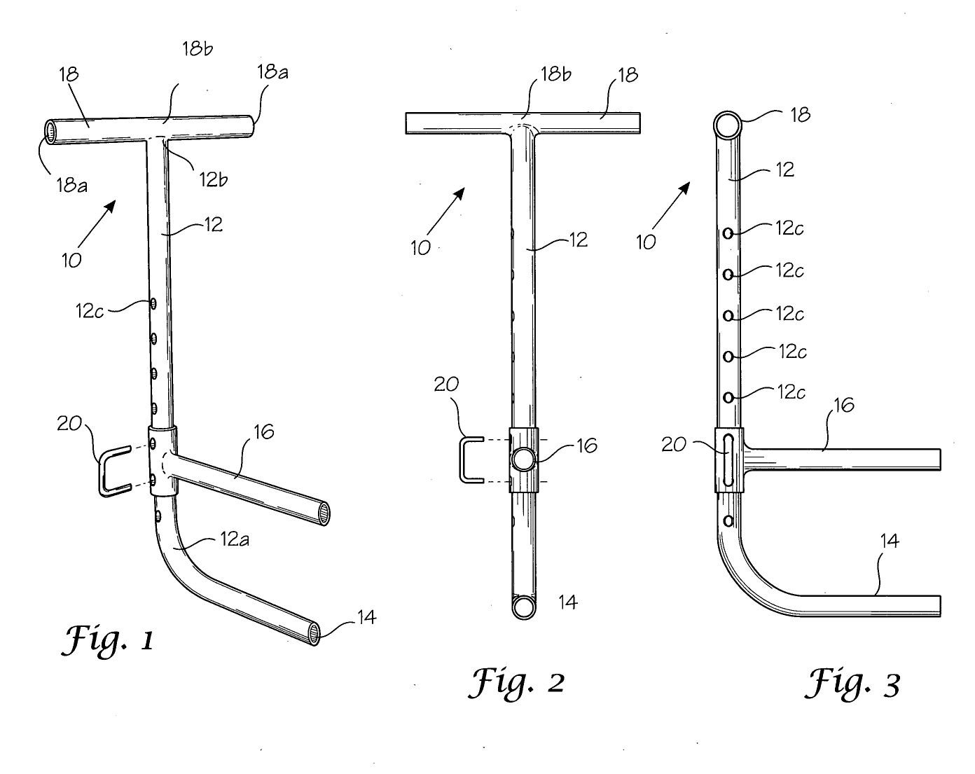

As seen in FIG. 1-3, a medical device 10 for reducing a fracture of the midshaft of a femur comprises a shaft 12, having a first 12a and a second end 12b , having a plurality of openings 12c running along the length of the shaft 12, a fixed arm 14 connected perpendicularly to the first end of the shaft 12, a movable arm 16 mounted on the shaft 12, wherein the movable arm 16 is parallel and immediately above the fixed arm 14, a lever 18, having two ends 18a and a midpoint 18b, the lever 18 is attached to the shaft 12 in a position that is perpendicular to the fixed arm 14, the shaft 12 attaches at the lever's midpoint 18b, and a locking means 20 for locking the movable arm 16 in a locked position.

The locking means 20 of the device 10 can be any known locking means in the art that can lock the movable arm 16 into a fixed position on the shaft 12. In a preferred embodiment, the locking means 20 is a pin 20 that slides into at least one of the openings 12c of the shaft 12.

The present invention is made of inflexible materials that can withstand sterilization, such materials are known in the medical art. In a preferred embodiment of the invention, the material will be anodized aluminum. Anodized aluminum is a preferred material of the inventor because it can be sterilized, it is inflexible, and it is not completely radio transparent. When the material is not completely radio transparent it has the inherent quality of permitting visualization of the placement of the arms in relation to the fracture site by image intensification.

The measurements of the device 10 are as follows: the shaft 14 is at least fourteen inches in length; the arms (14 & 16) are at least seven inches in length; and the lever's length 18 is sufficient to allow a surgeon to grasp the ends of the lever 18a and manipulate the device 10. In a preferred embodiment of the device 10, the measurements are as follows: the shaft 12 is twenty inches in length; the arms (14 & 16) are eleven inches in length; and the lever's length 18 is sufficient to allow a surgeon to grasp the ends of the lever 18b and manipulate the device 10.

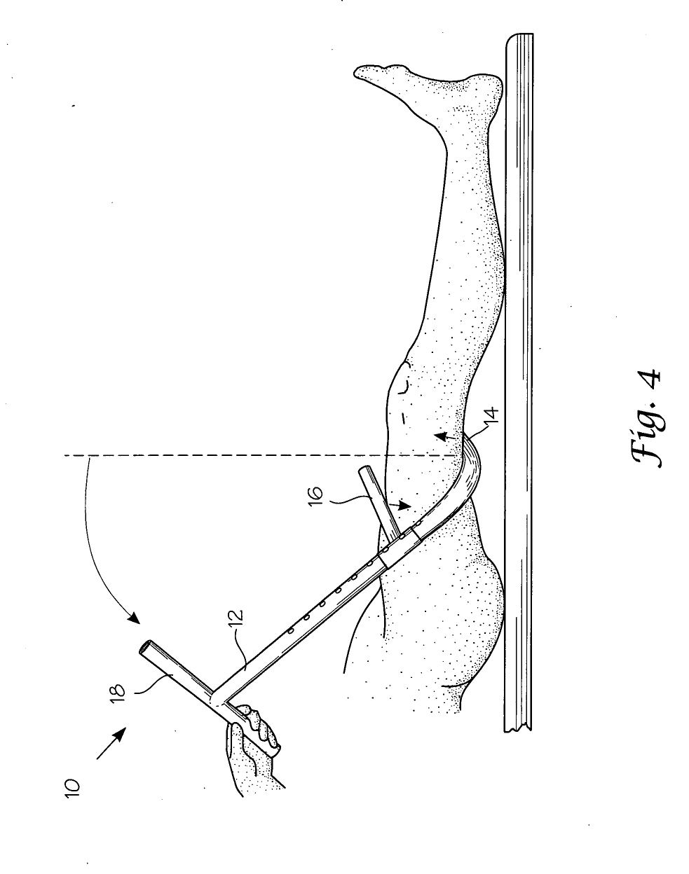

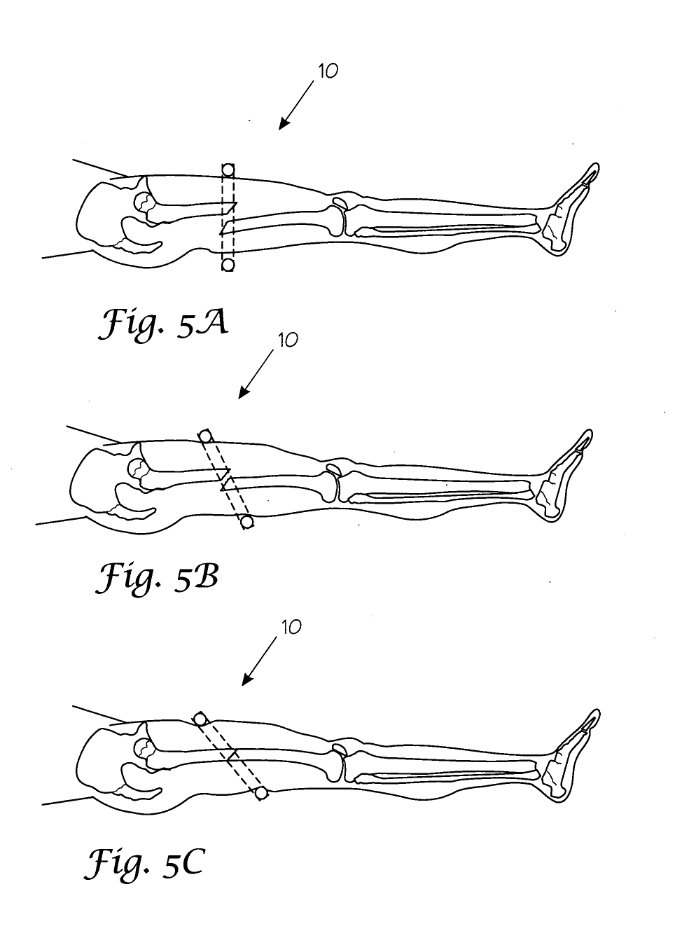

As seen in FIGS. 4-5C, the present invention is used by placing the device 10 externally at the level of the femoral fracture site. The device 10 is first placed on the patient so that the fixed arm 14 of the device 10 is placed at the thigh in a position that is posterior to the level of the fracture site and the movable arm 16 is placed in a position that is anterior to the level of the fracture site. Then the movable arm 16 of the device 10 is moved to a position that minimizes the distance between the fixed 14 and the movable arm 16 while embracing the thigh and then locking the movable arm 16 in that position. Then the surgeon will move the lever 18 of the device in a manner that permits him to exert pressure on the fractured ends of the femur, thereby aligning the fragments at the fracture site. After the surgeon aligns the fragments into an adequate position, he then performs the known steps required to insert the intramedullary rod into the medullary canal of the femur of the thigh.

When using the device, it is essential that the arms be placed in the closest proximity to each other, thereby increasing the pressure at the ends of the fracture fragments which in turn maximizes the alignment of the displaced fragments.

An advantage of this invention is that it permits a surgeon to align displaced femoral fragments, thereby permitting the insertion of an intramedullary rod without having to open the fracture site.

A further advantage of this invention is that it allows surgeons to align displaced femoral fragments without having to struggle when manipulating the femoral fragment. The present invention is a tool that uses a torque principle to reduce the amount of energy a surgeon has to exert when aligning the fractured ends of the femur.

Although the present invention has been described in considerable detail with reference to certain preferred versions thereof, other versions are possible. Therefore the spirit and the scope of the claims should not be limited to the description of the preferred versions contained herein.

What is claimed is:

1. A medical device for reducing a fracture of the midshaft of a femur which comprises:
a shaft, having a first and a second end, having a plurality of openings running along the length of the shaft:
a fixed arm connected perpendicularly to the first end of the shaft;
a movable arm mounted on the shaft, wherein the movable arm is parallel and immediately above the fixed arm;
a lever, having two ends and a midpoint, attached to the shaft in a position perpendicular to the fixed arm, the shaft attaches to the lever's midpoint; and
a locking means for locking the movable arm in a locked position.

2. The medical device of claim 1, wherein the locking means is a pin that slides into at least one of the openings of the shaft.

3. The medical device of claim 2, wherein the device is made of an inflexible material that can withstand sterilization.

4. The medical device of claim 3, wherein the material is anodized aluminum.

5. The medical device of claim 4, wherein the shaft is at least fourteen inches in length, the arms are at least seven inches in length, and the lever's length is sufficient to allow a user to grasp the ends of the lever and manipulate the device.

6. The medical device of claim 3, wherein the shaft is twenty inches in length, the arms are eleven inches in length, and the lever's length is sufficient to allow a user to grasp the ends of the lever and manipulate the device.

7. The medical device of claim 1, wherein the device is made of an inflexible material that can withstand sterilization.

8. The medical device of claim 7, wherein the material is anodized aluminum.

9. The medical device of claim 8, wherein the shaft is at least fourteen inches in length, the arms are at least seven inches in length, and the lever's length is sufficient to allow a surgeon to grasp the ends of the lever and manipulate the device.

10. The medical device of claim 09, wherein the locking means is a pin that slides into at least one of the openings of the shaft.

11. The medical device of claim 1, wherein the shaft is twenty inches in length, the arms are eleven inches in length, and the lever's length is sufficient to allow a surgeon to grasp the ends of the lever and manipulate the device.

12. The medical device of claim 11, wherein the device is made of an inflexible material that can withstand sterilization.

13. The medical device of claim 12, wherein the material is anodized aluminum.

14. The medical device of claim 13, wherein the locking means is a pin that slides into at least one of the openings of the shaft.

15. A method of using the device of claim 1, comprising the steps of:
placing the device on a patient so that the fixed arm of the device is placed at the thigh in a position that is posterior to the level of a fracture site and the movable arm is placed in a position that is anterior to the level of the fracture site;
moving the movable arm of the device to a position that minimizes the distance between the fixed and the movable arm while embracing the thigh of the patient;
locking the movable arm in the position that minimizes the distance between the fixed and the movable arm;
moving the lever of the device so that the fragments at the fracture site are aligned; and
inserting the intramedullary rod into the medullary canal of the femur of the thigh.
